(19) United States
(12) Patent Application Publication
(10) Pub. No.: US 2018/0146660 A1
(43) Pub. Date: May 31, 2018

(54) Use of Recombinant Human Activated Protein C to Enhance Viability of Transplant Tissue

(71) Applicant: University of Maryland, Baltimore, Baltimore, MD (US)

(72) Inventors: Donald G. Harris, Monona, WI (US); Agnes M. Azimzadeh, Timonium, MD (US); Richard N. Pierson III, Ruxton, MD (US)

(21) Appl. No.: 15/577,445

(22) PCT Filed: Jun. 3, 2016
(86) PCT No.: PCT/US16/35732

§ 371 Date: Nov. 28, 2017

Related U.S. Application Data
(60) Provisional application No. 62/171,661, filed on Jun. 5, 2015.

(51) Int. Cl. A01N 1/02; A61K 38/36; A61K 35/14; A61K 36/48

(52) U.S. Cl. CPC A01N 1/0226; A61K 38/36; A61K 2300/00; A61K 36/48; A61K 35/14

(57) ABSTRACT

The present invention relates to the use of activated Protein C (aPC) to enhance the viability of transplant graft. In certain instances, the invention provides methods of ex vivo perfusion of transplant graft with a composition comprising aPC.

### CROSS-REFERENCES TO RELATED APPLICATIONS

This application claims priority to U.S. Provisional Patent Application No. 62/171,661 filed on Jun. 5, 2015, the contents of which are incorporated by reference herein in its entirety.

### STATEMENT REGARDING FEDERALLY SPONSORED RESEARCH OR DEVELOPMENT

This invention was made with government support under Grant Nos. HL007698 and AI090959 awarded by the awarded by the National institutes of Health. The government has certain rights in the invention.

### BACKGROUND OF THE INVENTION

Organ transplantation is an effective treatment of end-stage organ failure, but is limited by a critical shortage of donor organs. Pigs are a promising source of organs, but pig-to-human xenotransplantation is limited by thrombotic inflammatory injury of the transplanted tissue. Wild type (WT) porcine xenografts are subject to hyperacute thrombotic injury, largely mediated by human antibodies against the porcine galactose 1,3α-galactose (Gal) antigen and complement activation. Xenografts from pigs genetically engineered to not express Gal, and are transgenic for the human complement regulatory protein CD46 (GalTKO.hCD46) have significantly improved survival, and are the genetic background for subsequent experimental genotypes (e.g. GalTKO.hCD46.hEPCR). However, despite these advances, activation of thrombosis remains a critical process contributing to xenograft injury.

A recent development has been pigs transgenic for human endothelial protein C receptor (hEPCR), which is intended to reduce xenograft thrombosis by promoting formation of activated protein C (aPC). aPC is an important endogenous anticoagulant, but it is not generated in unmodified pig xenografts. A secondary function of hEPCR is to serve as a receptor for aPC. In this capacity, the receptor stimulates intracellular pathways that protect cells from inflammation and external injury. While hEPCR can serve its first function (aPC catalysis) essentially instantaneously, the effects of its second function (cytoprotection) are dependent upon intracellular signaling pathways that take hours to lead to phenotypic changes.

Thus there is a need in the art for compositions and methods for improved survival of organ transplants. The present invention satisfies this unmet need.

### SUMMARY OF THE INVENTION

In one aspect, the present invention provides a method for improving the viability of transplant graft. The method comprises contacting the transplant graft with a composition comprising activated Protein C (aPC), an isolated nucleic acid molecule encoding aPC, Protein C (PC), an isolated nucleic acid molecule encoding PC, or a combination thereof. In one embodiment, the method reduces or prevents thrombosis in the transplant graft.

In one embodiment, the transplant graft is contacted with the composition ex vivo. In one embodiment, the transplant graft is allogeneic. In one embodiment, the transplant graft is xenogeneic. In one embodiment, the transplant graft is a lung, heart, kidney, liver, pancreas, intestine, multivisceral transplant, or a combination thereof.

In one embodiment, the transplant graft expresses Endothelial Protein C Receptor (EPCR). In one embodiment, the transplant graft is from an organism modified to express EPCR.

In one embodiment, the method comprises perfusing the transplant graft with the composition. In one embodiment, the composition comprises a crystalloid perfusate comprising aPC. In one embodiment, the composition comprises a whole-blood perfusate comprising aPC.

In one aspect, the present invention provides a method for improving the viability of transplant graft comprising a.) obtaining transplant graft; b.) contacting the transplant graft with a composition comprising activated Protein C (aPC), an isolated nucleic acid molecule encoding aPC, Protein C (PC), an isolated nucleic acid molecule encoding PC, or a combination thereof and c.) transplanting the transplant graft to a recipient.

In one aspect, the present invention provides a composition for improving the viability of transplant graft comprising activated Protein C (aPC), an isolated nucleic acid molecule encoding aPC, Protein C (PC), an isolated nucleic acid molecule encoding PC, or a combination thereof.

In one embodiment, the composition comprises a crystalloid perfusate. In one embodiment, the composition comprises a whole-blood perfusate.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A through FIG. 1F, depicts representative xenoperfusion images and 3D renderings, demonstrating assessment of xenogenic thrombus formation in a physiologic assay using live porcine endothelium, as would be present in a transplanted porcine xenograft. In all cases, porcine endothelium is perfused with human blood, and resultant platelet thrombus formation is visualized. FIG. 1A and FIG. 1B: wildtype (WT) endothelium. FIG. 1C and FIG. 1D: GalTKO endothelium. FIG. 1E and FIG. 1F: endothelium perfused with abciximab treated blood as a negative control demonstrating absence of platelet aggregation.

FIG. 2 is a graph depicting quantified data extracted from the source images in FIG. 1, demonstrating measurement of platelet adhesion to wildtype and GalTKO endothelia during porcine endothelial perfusion with human blood.

FIG. 3 is a set of representative xenoperfusion images and 3D renderings demonstrating that that the GalTKO.hCD46.hEPCR genotype (middle panel) reduces thrombus formation compared to the WT genotype (left panel). Thrombosis is further reduced by treating GalTKO.hCD46.hEPCR endothelium with aPC for 6 hours before and during the perfusion experiment (right panel).

FIG. 4 is a graph demonstrating that treating GalTKO.hCD46.hEPCR endothelium with aPC reduces adhesion compared to untreated GalTKO.hCD46.hEPCR.

FIG. 5 is a graph demonstrating that treating GalTKO.hCD46.hEPCR endothelium with aPC reduces aggregation compared to untreated GalTKO.hCD46.hEPCR.

FIG. 6 is a graph demonstrating that treating GalTKO.hCD46.hEPCR endothelium with aPC delays thrombus formation compared to untreated GalTKO.hCD46.hEPCR.

FIG. 7 is a graph demonstrating that aPC pretreatment is primarily responsible for the reduced thrombus burden achieved by aPC treatment, rather than a direct anticoagulant effect from its presence in the human blood during the perfusion. This demonstrates hEPCR's cytoprotective pathway is stimulated during the pre-treatment phase and is responsible for the treatment effect.

FIG. 8 is a table demonstrating that aPC effects are specific to hEPCR+tissue.

FIG. 9 is a graph from a separate assay of endothelial permeability, demonstrating that pretreatment of EPCR-expressing cells with aPC reduces thrombin-induced loss in cell barrier integrity by about 50% compared to untreated endothelium.

### DETAILED DESCRIPTION

The present invention relates to compositions and methods for improving the viability of transplant graft. For example, in certain embodiments, the invention relates to compositions and methods for suppressing inflammation and decreasing thrombosis in allogeneic and xenogeneic transplant grafts. In certain instances, the cytoprotection afforded by the present invention prolongs the viability of harvested graft, improves the survival of transplanted graft, and reduces rejection of transplanted graft.

This invention describes a new use for aPC to condition tissues expressing hEPCR (allogenic or xenogenic tissues) prior to a physiologic insult. Such pre-treatment of hEPCR expressing tissues with aPC enables activation of the cytoprotective intracellular pathways mediated by hEPCR in time for these changes to take effect by the time the tissues are exposed to inflammatory injury.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

An “effective amount” or “therapeutically effective amount” of a compound is that amount of a compound which is sufficient to provide a beneficial effect to the subject to which the compound is administered. An “effective amount” of a delivery vehicle is that amount sufficient to effectively bind or deliver a compound.

“Graft” refers to a cell, tissue, organ or otherwise any biological compatible substrate for transplantation.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

The terms “patient,” “subject,” “individual,” and the like are used interchangeably herein, and refer to any animal, or cells thereof whether in vitro or in vivo, amenable to the methods described herein. In certain non-limiting embodiments, the patient, subject or individual is a human.

“Allogeneic” refers to a graft derived from a different animal of the same species.

As used herein, the term “autologous” is meant to refer to any material derived from the same individual to which it is later to be re-introduced into the individual.

“Xenogeneic” refers to a graft derived from an animal of a different species.

“Isolated” means altered or removed from the natural state. For example, a nucleic acid or a peptide naturally present in its normal context in a living animal is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “conjugated” refers to covalent attachment of one molecule to a second molecule.

“Transplant” refers to a biocompatible substrate or a donor tissue, organ or cell, to be transplanted. An example of a transplant may include but is not limited to a tissue, a stem cell, a neural stem cell, a skin cell, bone marrow, and solid organs such as heart, pancreas, kidney, lung and liver.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to compositions and methods for improving the viability of transplant graft, reducing or preventing transplant graft damage, and enhancing survival of transplant graft. In certain instances, the cytoprotection afforded by the present invention prolongs the viability of harvested graft, improves the survival of transplanted graft, and reduces rejection of transplanted graft.

In one aspect, the present invention relates to a method of improving the viability of transplant graft by contacting the transplant graft with an effective amount of activated Protein C (aPC). For example, in certain embodiments, the method comprises perfusing the transplant graft with a solution comprising aPC.

The transplant graft may be any suitable cell, tissue, or organ to be transplanted into a recipient subject, including, but not limited to, lung, heart, kidney, liver, pancreas, intestine, multivisceral transplant (e.g., liver, stomach, duodenum, pancreases and small bowel), and portions thereof.

aPC is an endogenous enzyme that (a) suppresses inflammation by triggering cytoprotective mechanisms through binding human Endothelial Protein C Receptor (hEPCR) and (b) decreases thrombosis by degrading coagulation factors. The present invention provides methods for improving survival of transplanted grafts and organs by (a) pretreating (e.g., ex vivo) transplant graft with exogenous recombinant human aPC to stimulate these cytoprotective effects prior to subsequent exposure to a cellular or tissue insult. In certain embodiments, the transplant graft is allogenic or xenogeneic. In certain embodiments, the transplant graft is xenogeneic, wherein the xenogeneic graft is modified to express hEPCR. In certain embodiments, the method comprises continuing treatment to maintain the cytoprotective and anti-coagulant effects of hEPCR-aPC binding.

The present invention is based in part on the discovery that that pretreating hEPCR transgenic xenogeneic grafts with aPC before exposure to human blood decreases subsequent thrombosis. Such pretreatment and/or continuing treatment provides a valuable mechanism for improving the performance and survival of these organs in research models, eventual clinical trials and practice. The invention can be used in surgical fields involving the implantation of allogeneic donor transplant tissue, transplant grafts or tissues engineered to express hEPCR.

In one embodiment, the method comprises contacting transplant graft with a composition comprising aPC. In one embodiment, the method comprises contacting transplant graft with a composition comprising Protein C (PC), where PC is activated (e.g., cleaved) to form aPC. For example, in certain embodiments, the composition may comprise one or more components for activation of PC, including, but not limited to thrombin. In certain embodiments, PC is activated by one or more endogenous components of the transplant graft.

The present invention encompasses the use of PC or aPC mutants, fragments, homologs, or fusion peptides that retain the function of improving transplant graft viability.

Exemplary amino acid sequences for human PC is provided, for example in GenBank AAA60166.1 which is hereby incorporated by reference in its entirety. Various PC sequences are also provided for example, in WO 91/09960, WO 01/159084, WO 98/44000, EP 0323149, WO 00/66754, WO 99/63070, EP 0946715, WO 99/20767, WO 0066753, U.S. Pat. No. 5,453,373, U.S. Pat. No. 5,460,953, U.S. Pat. No. 5,270,178, U.S. Pat. No. 5,041,376, WO 01/57193, WO 01/36462, U.S. Pat. No. 4,775,624, U.S. Pat. No. 4,968,626, WO 03/106666, WO 04/044190, WO 04/113385, WO 06/044294, and U.S. Pat. No. 9,192,657 each of which is hereby incorporated by reference in its entirety. Recombinant aPC has been described (Bernard et al., 2001, N Engl J Med, 344: 699-709; which is hereby incorporated by reference in its entirety).

In one embodiment, the method comprises contacting the transplant graft with a composition comprising Drotrecogin alfa (XIGRIS®).

The invention should also be construed to include any form of a peptide having substantial homology to the peptides disclosed herein. Preferably, a peptide which is “substantially homologous” is about 50% homologous, more preferably about 70% homologous, even more preferably about 80% homologous, more preferably about 90% homologous, even more preferably, about 95% homologous, and even more preferably about 99% homologous to amino acid sequence of the peptides disclosed herein.

In one embodiment, the composition of the invention comprises a peptide, a fragment of a peptide, a homolog, a variant, a derivative or a salt of a peptide described herein. For example, in certain embodiments, the composition comprises a peptide comprising aPC, a fragment of aPC, a homolog of aPC, a variant of aPC, a derivative of aPC, or a salt of aPC. In certain embodiments, the composition comprises a peptide comprising PC, a fragment of PC, a homolog of PC, a variant of PC, a derivative of PC, or a salt of PC.

In certain embodiments, the peptide comprises a targeting domain, which targets the peptide to a desired location. For example, in certain embodiments, the targeting domain binds to a targeted cell, or protein thereby delivering the therapeutic peptide to a desired location. In certain embodiments, the targeting domain comprises a peptide, nucleic acid, small molecule, or the like, which has the ability to bind to the targeted cell or protein. For example, in one embodiment, the targeting domain comprises an antibody or antibody fragment which binds to a targeted cell or protein.

The peptide of the present invention (e.g., aPC, PC, and variants thereof, etc.) may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. The composition of a peptide may be confirmed by amino acid analysis or sequencing.

The variants of the peptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present invention, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation. A variety of approaches are available for introducing unnatural amino acids during protein translation. By way of example, special tRNAs, such as tRNAs which have suppressor properties, suppressor tRNAs, have been used in the process of site-directed non-native amino acid replacement (SNAAR). In SNAAR, a unique codon is required on the mRNA and the suppressor tRNA, acting to target a non-native amino acid to a unique site during the protein synthesis (described in WO90/05785). However, the suppressor tRNA must not be recognizable by the aminoacyl tRNA synthetases present in the protein translation system. In certain cases, a non-native amino acid can be formed after the tRNA molecule is aminoacylated using chemical reactions which specifically modify the native amino acid and do not significantly alter the functional activity of the aminoacylated tRNA. These reactions are referred to as post-aminoacylation modifications. For example, the epsilon-amino group of the lysine linked to its cognate tRNA (tRNA_{LYS}), could be modified with an amine specific photoaffinity label.

The peptides of the invention may be conjugated with other molecules, such as proteins, to prepare fusion proteins. This may be accomplished, for example, by the synthesis of N-terminal or C-terminal fusion proteins provided that the resulting fusion protein retains the functionality of the peptide of the invention.

Cyclic derivatives of the peptides the invention are also part of the present invention. Cyclization may allow the peptide to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulphide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

The peptides of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

Peptides of the invention may also have modifications. Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are peptides which have been modified using ordinary molecular biological techniques so as to improve their resistance to proteolytic degradation or to optimize solubility properties or to render them more suitable as a therapeutic agent. Such variants include those containing residues other than naturally-occurring L-amino acids, e.g., D-amino acids or non-naturally-occurring synthetic amino acids. The peptides of the invention may further be conjugated to non-amino acid moieties that are useful in their therapeutic application. In particular, moieties that improve the stability, biological half-life, water solubility, and/or immunologic characteristics of the peptide are useful. A non-limiting example of such a moiety is polyethylene glycol (PEG).

Covalent attachment of biologically active compounds to water-soluble polymers is one method for alteration and control of biodistribution, pharmacokinetics, and often, toxicity for these compounds (Duncan et al., 1984, Adv. Polym. Sci. 57:53-101). Many water-soluble polymers have been used to achieve these effects, such as poly(sialic acid), dextran, poly(N-(2-hydroxypropyl)methacrylamide) (PHPMA), poly(N-vinylpyrrolidone) (PVP), poly(vinyl alcohol) (PVA), poly(ethylene glycol-co-propylene glycol), poly(N-acryloyl morpholine (PAcM), and poly(ethylene glycol) (PEG) (Powell, 1980, Polyethylene glycol. In R. L. Davidson (Ed.) Handbook of Water Soluble Gums and Resins. McGraw-Hill, New York, chapter 18). PEG possess an ideal set of properties: very low toxicity (Pang, 1993, J. Am. Coll. Toxicol. 12: 429-456) excellent solubility in aqueous solution (Powell, supra), low immunogenicity and antigenicity (Dreborg et al., 1990, Crit. Rev. Ther. Drug Carrier Syst. 6: 315-365). PEG-conjugated or “PEGylated” protein therapeutics, containing single or multiple chains of polyethylene glycol on the protein, have been described in the scientific literature (Clark et al., 1996, J. Biol. Chem. 271: 21969-21977; Hershfield, 1997, Biochemistry and immunology of poly(ethylene glycol)-modified adenosine deaminase (PEG-ADA). In J. M. Harris and S. Zalipsky (Eds) Poly(ethylene glycol): Chemistry and Biological Applications. American Chemical Society, Washington, D.C., p 145-154; Olson et al., 1997, Preparation and characterization of poly(ethylene glycol)ylated human growth hormone antagonist. In J. M. Harris and S. Zalipsky (Eds) Poly(ethylene glycol): Chemistry and Biological Applications. American Chemical Society, Washington, D.C., p 170-181).

A peptide of the invention may be synthesized by conventional techniques. For example, the peptides of the invention may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis.)

The peptides may be chemically synthesized by Merrifield-type solid phase peptide synthesis. This method may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., 2000, Ann. Rev. Biochem. 69:923-960). An advantage to the utilization of a synthetic peptide route is the ability to produce large amounts of peptides, even those that rarely occur naturally, with relatively high purities, i.e., purities sufficient for research, diagnostic or therapeutic purposes.

Solid phase peptide synthesis is described by Stewart et al. in Solid Phase Peptide Synthesis, 2nd Edition, 1984, Pierce Chemical Company, Rockford, Ill.; and Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the alpha-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and coupling thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group, such as formation into a carbodiimide, a symmetric acid anhydride, or an “active ester” group, such as hydroxybenzotriazole or pentafluorophenyl esters.

Examples of solid phase peptide synthesis methods include the BOC method which utilized tert-butyloxcarbonyl as the alpha-amino protecting group, and the FMOC method which utilizes 9-fluorenylmethyloxcarbonyl to protect the alpha-amino of the amino acid residues, both which methods are well-known by those of skill in the art.

Incorporation of N- and/or C-blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function, e.g. with DCC, can then proceed by addition of the desired alcohol, followed by de-protection and isolation of the esterified peptide product.

The peptides of the invention may be prepared by standard chemical or biological means of peptide synthesis. Biological methods include, without limitation, expression of a nucleic acid encoding a peptide in a host cell or in an in vitro translation system.

Included in the invention are nucleic acid sequences that encode the peptide of the invention. In one embodiment, the invention includes nucleic acid sequences encoding the amino acid sequence of aPC or PC. Accordingly, subclones of a nucleic acid sequence encoding a peptide of the invention can be produced using conventional molecular genetic manipulation for subcloning gene fragments, such as described by Sambrook et al., Molecular Cloning: A Laboratory Manual, Cold Springs Laboratory, Cold Springs Harbor, N.Y. (2012), and Ausubel et al. (ed.), Current Protocols in Molecular Biology, John Wiley & Sons (New York, N.Y.) (1999 and preceding editions), each of which is hereby incorporated by reference in its entirety. The subclones then are expressed in vitro or in vivo in bacterial cells to yield a smaller protein or polypeptide that can be tested for a particular activity.

In certain embodiments, the method comprises contacting transplant graft with a composition comprising an isolated nucleic acid molecule encoding aPC, PC, or a variant thereof.

The nucleotide sequence of the isolated nucleic acids include both the DNA sequence that is transcribed into RNA and the RNA sequence that is translated into a polypeptide. According to other embodiments, the nucleotide sequences are inferred from the amino acid sequence of the peptides of the invention. As is known in the art several alternative nucleotide sequences are possible due to redundant codons, while retaining the biological activity of the translated peptides.

Further, the invention encompasses an isolated nucleic acid comprising a nucleotide sequence having substantial homology to a nucleotide sequence encoding a disclosed herein. Preferably, the nucleotide sequence of an isolated nucleic acid is “substantially homologous,” that is, is about 60% homologous, more preferably about 70% homologous, even more preferably about 80% homologous, more preferably about 90% homologous, even more preferably, about 95% homologous, and even more preferably about 99% homologous to a nucleotide sequence of an isolated nucleic acid encoding a peptide of the invention.

Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York).

The desired nucleic acid encoding aPC, PC, or a variant thereof can be cloned into a number of types of vectors. However, the present invention should not be construed to be limited to any particular vector. Instead, the present invention should be construed to encompass a wide plethora of vectors which are readily available and/or well-known in the art. For example, a desired polynucleotide of the invention can be cloned into a vector including, but not limited to a plasmid, a phagemid, a phage derivative, an animal virus, and a cosmid. Vectors of particular interest include expression vectors, replication vectors, probe generation vectors, and sequencing vectors.

In specific embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Further, the expression vector may be provided in the form of a viral vector. Viral vector technology is well known in the art and is described, for example, in Sambrook et al. (2012), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to the transplant graft. A number of retroviral systems are known in the art. In some embodiments, adenovirus vectors are used. A number of adenovirus vectors are known in the art. In one embodiment, lentivirus vectors are used.

For expression of the desired polynucleotide, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, i.e., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. No. 4,683,202, U.S. Pat. No. 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous. In one embodiment, the promoter or enhancer specifically directs expression of aPC, PC, or variant thereof in the transplant graft.

In order to assess the expression of the desired polynucleotide, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of siRNA polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In one aspect, the present method comprises contacting the transplant graft with composition comprising a cell which expresses aPC, PC, or a variant thereof. In one embodiment, the cell is genetically modified to express a protein and/or nucleic acid described herein. In certain embodiments, genetically modified cell is autologous to the transplant recipient. Alternatively, the cells can be allogeneic, syngeneic, or xenogeneic with respect to the recipient. In certain embodiment, the cell is able to secrete or release the expressed protein in order to deliver the peptide to the transplant graft.

The genetically modified cell may be modified, using techniques standard in the art. Genetic modification of the cell may be carried out using an expression vector or using a naked isolated nucleic acid construct.

In one embodiment, the cell is obtained and modified ex vivo, using an isolated nucleic acid encoding one or more proteins described herein. In one embodiment, the cell is obtained from a recipient, genetically modified to express the protein and/or nucleic acid, and is then placed in contact or in the vicinity of transplant graft. In certain embodiments, the cell is expanded ex vivo or in vitro to produce a population of cells. In certain embodiments, the transplant graft is placed in a container comprising a suitable media and a cell which expresses aPC, PC, or variant thereof.

In certain embodiments, the composition comprising aPC, PC, or variant thereof nucleic acid encoding aPC, PC, or variant thereof or cell expressing aPC, PC or variant thereof is administered to the transplant graft following removal or harvest of the graft from a donor. For example, in certain embodiments, the composition is administered to the transplant graft less than 72 hours, 48 hours, 36 hours, 24 hours, 18 hours, 12 hours, 6 hours, 5 hours, 4 hours, 3 hours, 2 hours, 1 hour, 45 minutes, 30 minutes, 15 minutes, 10 minutes, 5 minutes, or 1 minute following removal or harvest of the graft from a donor. In certain embodiments, the composition is administered to the transplant graft for more than 72 hours, 48 hours, 36 hours, 24 hours, 18 hours, 12 hours, 6 hours, 5 hours, 4 hours, 3 hours, 2 hours, 1 hour, 45 minutes, 30 minutes, 15 minutes, 10 minutes, 5 minutes, or 1 minute prior to transplantation of the transplant graft into the rceipient.

In certain embodiments, the transplant graft is allogeneic. For example, in certain embodiments, where the recipient is a human, the donor from which the graft transplant is harvested is a human. For example, in certain embodiments, the donor is characterized by brain death. In certain embodiments, “brain death” is defined as the total cessation of brain function, including brain stem function, e.g., wherein there is no oxygen or blood flow to the brain, or wherein the brain no longer functions in any manner and will never function again.

In certain embodiments, the donor is not diagnosed as having a chronic, transmissible, or infectious physical ailment, e.g., for which pharmacological intervention is or would have been suitable. In certain embodiments, the donor is not currently and/or has not been diagnosed with diabetes, cancer, high blood pressure, kidney disease, or cardiovascular disease, e.g., atherosclerosis or heart disease.

In certain embodiments, the transplant graft is xenogeneic. For example, in certain embodiments, where the recipient is a human, the donor is a non-human animal. For example, the donor may be a pig or primate, such as a genetically altered animal. In certain such embodiments, the donor is an animal that has been genetically modified such that proteins on the surface of the animal's organs and/or cells are recognized as compatible by a human immune system. For example, the donor may be an animal that has been genetically modified such that proteins on the surface of the animal's organs and/or cells are recognized as human by the human immune system, so the transplant graft is not attacked when transplanted. For example, the donor may be an animal that has been genetically modified to express hEPCR. In certain embodiments, the donor is a pig.

The transplant graft may be any suitable cell, tissue, or organ to be received by the recipient. Exemplary types of transplant graft includes, but is not limited to, lung, heart, kidney, liver, pancreas, intestine, multivisceral transplant (e.g., liver, stomach, duodenum, pancreases and small bowel), and portions thereof.

In certain embodiments, the method comprises contacting the transplant graft with a preservation solution wherein the preservation solution comprises aPC, PC, or variant thereof; a nucleic acid molecule encoding aPC, PC, or variant thereof or a cell expressing aPC, PC, or variant thereof. In certain embodiments, the preservation solution comprises aPC, PC, or a variant thereof at a concentration of 0.1 ng/mL to 1 gram/mL.

In certain embodiments, the organ preservation solution further comprises potassium, sodium, magnesium, calcium, phosphate, sulphate, glucose, citrate, mannitol, histidine, tryptophan, alpha-ketoglutaric acid, lactobionate, raffinose, adenosine, allopurinol, glutathione, glutamate, insulin, dexamethasone, hydroxyethyl starch, bactrim, trehalose, gluconate, or combinations thereof. In certain embodiments, the preservation solution comprises sodium, potassium, magnesium, or combinations thereof. In certain embodiments, the preservation solution is free or substantially free of cells, coagulation factors, nucleic acids such as DNA, and/or plasma proteins. In certain embodiments, the preservation solution is sterile. In certain embodiments, organ preservation solution comprises an aqueous solution. In certain embodiments, the preservation solution comprises a perfluorocarbon, such as a perfluoro hydrocarbon or a perfluoroalkylamine. Exemplary perfluorocarbons are described in Transplantation, 74(12), 1804-1809, Dec. 27, 2002 and Am. Assoc. of Nurse Anesthetists Journal, 74(3): 205-211, June 2007, the compounds in which are incorporated herein by reference. The preservation solution may be any suitable preservation solution known in the art. Examples of such preservation solutions include, but are not limited to, University of Wisconsin solution, Krebs-Henseleit solution, Celsior solution, St. Thomas Hospital 2 solution, Ringer-lactate solution, Collins solution, Euro-Collins solution, Stanford solution, Ross-Marshall citrate solution, phosphate-buffered sucrose solution, Kyoto ET solution, or Bretschneider histidine tryptophan ketoglutarate (HTK) solution.

The transplant graft may be contacted with (or administered) the composition comprising aPC, PC, or variant thereof; a nucleic acid molecule encoding aPC, PC, or variant thereof; or a cell expressing aPC, PC, or variant thereof at any point during the transplantation process. For example, the composition may be administered by flushing the transplant graft, continuously perfusing the transplant graft, or intermittently perfusing through the blood vessels of the transplant graft while the transplant graft is still in a donor's body, during the removal of the transplant graft from a donor's body, after the transplant graft is removed from a donor's body, while the transplant graft is being transplanted into a recipient, immediately after the transplant graft is transplanted into a recipient, or any combination thereof.

In certain embodiments, the composition comprising aPC, PC, or variant thereof; a nucleic acid molecule encoding aPC, PC, or variant thereof; or a cell expressing aPC, PC, or variant thereof is a crystalloid perfusate or a whole-blood perfusate. In certain embodiments, the whole-blood perfusate is blood-type matched of the recipient. In certain embodiments, the whole-blood perfusate is of the recipient.

In certain embodiments, the composition further comprises one or more additional agents that aid in the viability or survival of the transplant graft. Exemplary additional agents include, but is not limited to, anti-inflammatories, anti-coagulants, anti-thrombotics, thrombolytics, anti-platelets, hormones, vitamins, and the like.

The perfusion of the transplant graft may be conducted using any methodology or equipment known in the art. Various aspects of perfusion of transplant graft can be found in Sanchez et al., 2012, J Heart Lung Transplant, 31(4): 339-48. Exemplary perfusion equipment includes, but is not limited to the Xvivo EVLP circuit, TransMedics Organ Care System, custom designed perfusion circuits (such as in Burdorf et al., 2014, Am J Transplantation, 14(5): 1084-1095), and other organ perfusion systems. In certain embodiments, for intestinal grafts, the superior mesenteric artery, celiac artery, or inferior mesenteric artery are cannulated and perfused with the composition described herein, and effluent is captured in a portal vein cannula or gravity drainage from an open venous system. In certain embodiments, for a composite tissue graft, the arterial supply is individually cannulated and perfused with the composition described herein, and effluent captured in a vein cannula, or by gravity drainage from an open venous system.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

Platelet activation and thrombin generation remain implicated in dysfunction of porcine lung xenografts, despite successive genetic modifications to control acute xenogeneic thrombosis. Human endothelial protein C receptor (hEPCR) regulates thrombotic and inflammatory pathways, and lungs from hEPCR transgenic pigs have improved performance versus the GalKO.hCD46 background in pre-clinical models (Budorf et al., 2013, The Journal of Heart and Lung Transplantation, 32(4): S137). Here, it was examined whether recombinant human activated Protein C (aPC) can modulate the thrombotic phenotype of hEPCR expressing cells.

Thrombosis was evaluated using a novel cellular xenoperfusion assay. Wild type (WT), GalKO.hCD46 and GalKO.hCD46.hEPCR porcine aortic endothelial cells were cultured in microfluidic channels. Perfusions were performed at 5 dynes/cm² with heparinized human blood stained with calcein-AM to label platelets. After confluent growth, control endothelia were incubated with media for 6 hours and perfused with blood (n=24). Treated channels were incubated with 0.02 μg/mL aPC (Haematologic Technologies) in media for 6 hours and perfused with blood+0.02 μg/mL aPC (n=17). Untreated WT endothelia (n=13) served as positive controls of thrombosis, while WT with (n=5) and GalKO.hCD46+/−(n=10 each) aPC controlled for treatment effect. Thrombosis was captured by serial fluorescent imaging and analyzed by percent surface area coverage (SA, %), time to 50% peak SA (T50, minutes) and the fluorescent intensity: SA ratio (FR, arbitrary units) as markers of adhesion, thrombosis kinetics and aggregation, respectively. Mean peak values were compared by unpaired Student's t-test.

WT channels had diffuse (SA: 66+/−17%) and rapid (T50: 17+/−10 min) adhesion, and extensive aggregation (FR: 112+/−4 a.u.), reflecting high-grade thrombosis. GalKO.hCD46 moderately decreased adhesion (SA 48+/−20, p=0.03) but did not slow adhesion (T50: 14+/−8, p=0.48) or suppress aggregation (FR: 123+/−3, p=0.35). Compared to GalKO.hCD46, the addition of the hEPCR transgene without additional treatment did not further affect thrombosis (SA: 42+/−18, p=0.40; T50: 18+/−12, p=0.31; FR: 118+/−23, p=0.57). However, aPC treatment reduced (SA: 15+/−10 p<0.0001) and delayed adhesion (T50: 26+/−7, p=0.04), and attenuated platelet aggregation (FR: 81+/−40, p<0.001) relative to untreated GalKO.hCD46.hEPCR endothelia. In contrast, aPC treatment did not affect thrombosis on WT or GalKO.hCD46 endothelia.

The described experiments demonstrate that in a physiologic cellular xenoperfusion assay, treatment with recombinant human aPC uniquely modulated the thrombotic phenotype of GalKO.hCD46.hEPCR porcine endothelium. Specifically, aPC further reduced and delayed platelet adhesion and suppressed platelet aggregation, beyond that achieved by transgenic modification. This effect was unique to hEPCR cells, and may provide a mechanism to enhance the performance of xenografts expressing hEPCR.

### Example 2

Current xenotransplantation models provide limited ability to investigate the mechanisms of xenogeneic thrombosis under physiologic conditions. Ex-vivo xenoperfusion is a valuable model of whole-organ, pig-to-human transplantation but does not enable dynamic characterization of events occurring at the interface between the xenograft endothelium and human blood. In contrast, standard static in-vitro assays lack physiologic shear-flow, which is a critical condition for a range of interactions occurring on the endothelium, including thrombosis.

Shear-flow perfusion systems enable dynamic study of thrombosis under physiologic conditions. However, most such models to date have utilized ligand-coated substrates, while live-cell studies with endothelia typically require intra-vital experiments with limited the ease and complexity. Further, such a platform has not been used to study xenogeneic thrombosis, which involves unique mechanisms that are still being defined. Described herein is the development and validation of a novel, cellular perfusion assay to model xenogeneic thrombosis under physiologic shear flow conditions, and demonstrate its initial application to studying xenogeneic thrombosis.

The materials and methods employed in these experiments are now described.

Instrumentation.

Experiments were performed using a Bioflux 1000 system (Fluxion Biosciences, South San Francisco) coupled with an automated AxioObserver Z1 microscope (Zeiss, Oberkochen). 24 channel low-shear microfluidic plates (Fluxion Biosciences) were used; each channel consists of a proximal inflow well that flows via a short-segment channel to a perfusion chamber (length 4.8 mm, width 0.35 mm, height 0.07 mm), which subsequently flows to an outflow well. The system is closed, and perfusate does not recirculate. Perfusion and imaging sequences were controlled using Bioflux system software from a standard desktop computer.

Animal Care and Use.

Except as noted, porcine descending thoracic aortae were removed after heart-lung explantation and used for primary cell culture.

Cell Culture.

Primary WT porcine aortic endothelial cells (PAEC), WT and GalTKO cell line PAEC were obtained from Immerge Biotherapeutics (Boston, Mass.). GalTKO.hCD46 and GalTKO.hCD46.hEPCR PAEC were cultured as previously described.

Standard culture media was Dulbeccos modified Eagles medium (Gibco-Life Technologies, Grand Island) supplemented with 10% heat-inactivated fetal bovine serum (Atlanta Biological, Lawrenceville), gentamicin (Gibco), amphotericin B, and endothelial cell growth supplement (Becton Dickinson, San Jose) (standard medium). Cells were grown in uncoated 75 cm² flasks incubated at 37° C. in 5% CO₂ and 83% relative humidity (standard incubation conditions), and split approximately every 3 days. Endothelial morphology was verified at each passage. Cells were used at the 4^{th} to 8^{th} passage by trypsinization (0.25% trypsin-EDTA; Gibco) and resuspension at 10 million cells/mL media.

Channels were coated with 100 μg/mL bovine plasma fibronectin (Sigma-Aldrich, St. Louis) in phosphate buffered solution (PBS) perfused at 5 dynes/cm² for 5 minutes. After 1 hour at room temperature, the channels were washed with culture medium at 5 dynes/cm² for 10 minutes. PAECs were seeded by bolusing cell suspension at 0.5 dynes/cm² into the channels under brightfield microscopic visualization, followed by a 60 second pause for initial attachment. Additional boluses were delivered to concentrate the cells to approximately 100-150/100× field of view.

The channels were incubated for 1 hour, and excess PAECs in the inflow well removed with two cycles of washing with 400 μL sterile double distilled H₂O plus incubation with 150 μL trypsin for 5 minutes. Media was introduced to the wells and allowed to nourish the channels by gravity flow during incubation to 100% confluence over 48-72 hours. Excess media was aspirated from wells immediately prior to experiments.

Perfusate Preparation and Perfusion Conditions.

Fresh whole blood was collected from a healthy volunteer by venipuncture into heparinized (18.75 IU/mL) vacutainers (Becton Dickinson). High dose anticoagulation was verified by pre- and post-perfusion activated clotting times, and physiologic blood cell counts were confirmed by an automated counter. Blood aliquots were stained with 0.5 μM calcein-AM and treated with test reagent or PBS vehicle. Aliquots were light protected and mixed by rocking for 30 minutes at room temperature. 350 μL of prepared blood was introduced into the inflow wells of experimental channels, and perfused at 5 dynes/cm² and 37° C. for 50 minutes. The most proximal 100× field of view of each channel's perfusion chamber was imaged every 30 seconds by FITC fluorescent microscopy with a 100 millisecond exposure. Typically, 12 channels were perfused and acquired simultaneously: 2 positive controls, 5 study controls and 5 experimental channels.

Data Analysis.

The sequential source images were stacked for their respective channels and analyzed using a component of Montage (Fluxion Biosciences) based on the Metamorph engine (Molecular Devices, Sunnyvale). To capture the most physiologically significant event, the signal threshold for each stack was determined from the image with the most surface area coverage by thrombus. Thresholds were highly consistent within and between experiments.

Surface area coverage (SA, percent) and fluorescent intensity (in arbitrary units) were extracted and exported to Excel (Microsoft, Redmond). SA was used directly as a measure of adhesion in the x and y dimensions between the endothelium and perfused platelets and leukocytes. The fluorescence: SA ratio (FR, in arbitrary units) determined relative binding in the z dimension, and was used as an index of platelet and leukocyte aggregation. Binding kinetics were measured by the time to 50% peak SA (T₅₀, minutes). Mean peak SA and FR, and T₅₀ values were compared by unpaired t-test. Results were qualitatively confirmed by 3D surface rendition using ImageJ (National Institutes of Health, Bethesda).

The results of the experiments are now described.

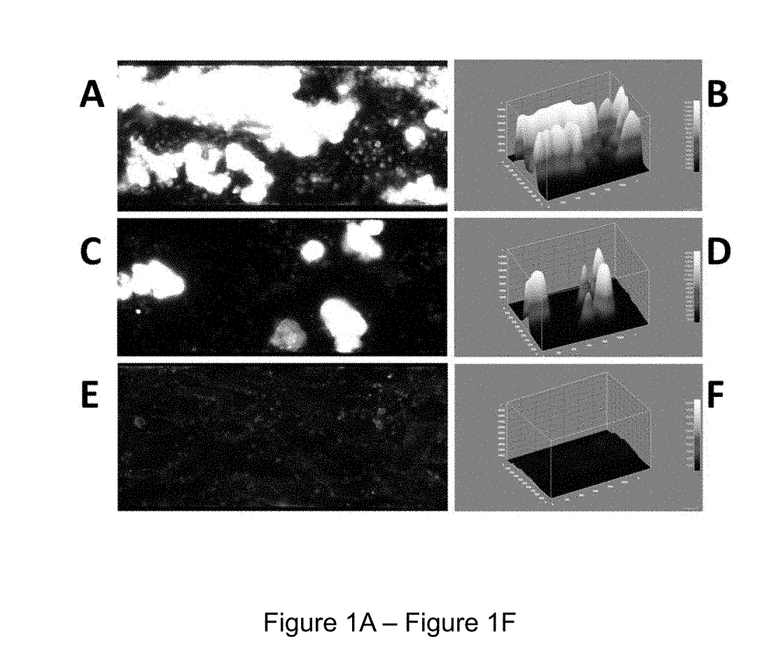

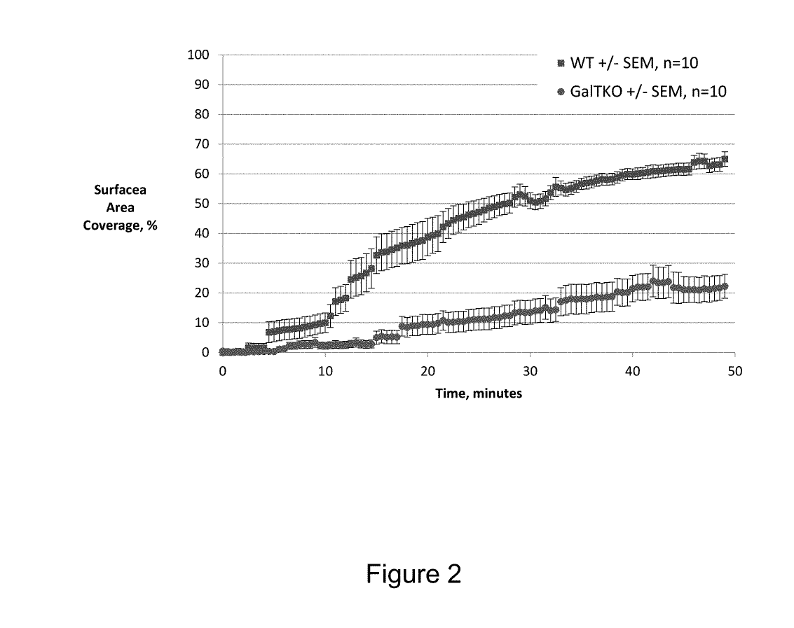

Initial experiments were performed by perfusing WT PAECs with heparinized human blood to recapitulate hyperacute thrombosis, and develop a positive control condition. Despite activated clotting times >999 seconds, WT endothelia rapidly stimulated diffuse adhesion and aggregation (n=50; Table 1 and FIG. 1A and FIG. 1B), indicating consistent, high-grade thrombosis. Thrombus formation occurred on a similar time course as thrombin formation and platelet activation and sequestration observed during ex-vivo WT lung xenoperfusion. Finally, consistent with established xenotransplantation models, thrombosis was decreased and delayed on GalTKO endothelia (n=11; Table 2, FIG. 1C, FIG. 1D, and FIG. 2), primarily from diminished adhesion as aggregation was only minimally affected.

To exclude platelet and coagulation activation by the system itself, post-perfusion images of the inflow reservoirs were obtained and displayed no significant thrombi. Similarly, perfusion of fresh whole GalTKO.hCD46.hEPCR blood on syngeneic endothelia resulted in no significant thrombosis (n=6, Table 2). In contrast, GalTKO.hCD46.hEPCR blood perfused on WT endothelia stimulated high-grade thrombosis similar to positive controls.

The FR as a measure of aggregation was tested by perfusing WT endothelia with human blood treated with, an anti-gp2b3a Fab (0.5 μg/mL abciximab, n=5). Abciximab had a minor effect on adhesion or binding kinetics, but substantially reduced fluorescence, validating FR (Table 1, FIG. 1E and FIG. 1F). In contrast, thrombin inhibition with bivalirudin (0.2 μg/mL, n=9; Table 1) substantially diminished adhesion, but had minimal effect on aggregation.

As described herein, this novel, cellular xenoperfusion assay enabled dynamic and mechanistic characterization of xenogeneic thrombosis. Consistent with established models, WT endothelia stimulated intense thrombus formation despite high-dose anticoagulation, and thrombosis was decreased by the GalTKO genotype. Validation and control experiments confirmed these results were specific to xenoperfusion. Interestingly, the model demonstrated that reduced thrombosis on GalTKO endothelia was due to decreased and delayed adhesion, while aggregation remained intact. This new finding suggests that future genetic modifications should target pathways contributing to platelet-platelet binding.

This model incorporated important physiologic conditions, include live-cell confluent endothelia, fresh whole blood perfusate and shear-flow similar to low-resistance capillaries that account for most endothelial-blood contact. This makes the assay suitable for both definitive in-vitro studies and as an advanced platform before proceeding to more resource intensive ex-vivo, in-vivo or intra-vital experimentation. In the xenotransplantation field, the assay enables screening candidate genotypes, testing anticoagulation protocols, or performing novel perfusion regimens prior to their application in ex-vivo organ xenoperfusion.

This model was specifically developed to model xenogeneic thrombosis, which involves thromboregulatory dysfunction distinct from innate processes. However, as the final common pathways of the coagulation cascade and platelet activity remain intact, the model may be useful for other experiments studying high-intensity thrombosis. Alternatively, the endothelial substrate could be tailored for non-xenoperfusion conditions. General advantages of the assay as developed, and the system in general, include the ability to perform high-throughput, physiologic experiments with minimal resources, and subsequently mechanistically dissect the component processes of thrombus formation.

TABLE 1
Human blood xenoperfusion results.

| Endothelia | | | | | | | |
|---|---|---|---|---|---|---|---|
| Genotype | n | SA, % | P | FR, a.u. | P | T₅₀, min. | P |
| WT (control) | 50 | 63.4 ± 13.8 | | 120.0 ± 7.0 | | 21.5 ± 11.6 | |
| GalTKO | 11 | 27.5 ± 13.9 | <0.0001 | 107.0 ± 15.3 | <0.0001 | 31.0 ± 9.4 | 0.01 |
| WT + | 5 | 49.6 ± 9.1 | 0.04 | 16.2 ± 4.0 | <0.0001 | 22.4 ± 4.9 | 0.87 |
| abciximab | | | | | | | |
| WT + | 9 | 25.4 ± 21.1 | <0.0001 | 112.7 ± 9.9 | 0.01 | 29.8 ± 15.4 | 0.08 |
| bivalirudin | | | | | | | |

SA, percent surface area coverage;
FR, fluorescence ratio;
T₅₀, time to 50% maximal surface area coverage.

TABLE 2
Porcine blood syngeneic and allo-incompatible perfusion results.

| Endothelia | | | | | | | |
|---|---|---|---|---|---|---|---|
| Genotype | n | SA, % | P | FR, a.u. | P | T₅₀, min. | P |
| WT | 4 | 67.2 ± 16.7 | | 122.4 ± 2.1 | | 29.5 ± 13.7 | |
| GalTKO.hC | 6 | 17.3 ± 6.0 | 0.0003 | 28.4 ± 8.2 | <0.0001 | 26.6 ± 12.3 | 0.76 |
| D46.hEPCR | | | | | | | |

SA, percent surface area coverage;
FR, fluorescence ratio;
T₅₀, time to 50% maximal surface area coverage.

### Example 3

Experiments were conducted to evaluate the effects of aPC treatment of transgenic porcine cells.

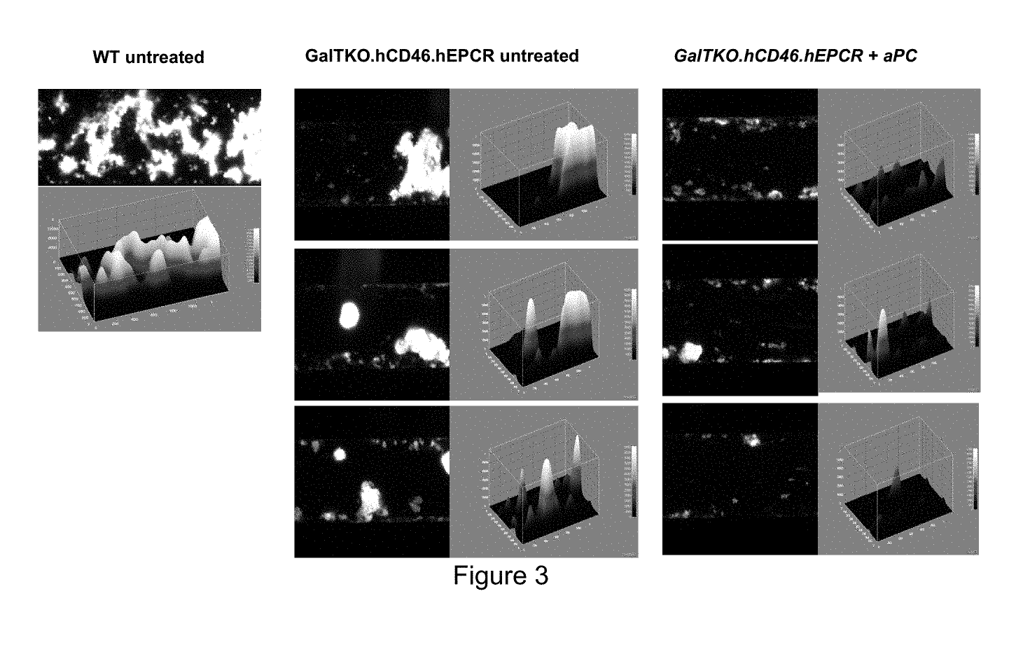

First, experiments were conducted to evaluate if aPC treatment affected thrombosis. GalTKO.hCD46.hEPCR porcine aortic endothelial cells were cultured on channels as previously described, and left untreated or treated with 0.02 μg/mL aPC for 6 hours prior to xenoperfusion. Channels were perfused as previously described and xenoperfusion images were obtained and 3D renderings were produced. It was observed that treatment with aPC reduced thrombosis (FIG. 3) as compared to untreated GalTKO.hCD46.hEPCR and untreated WT cells.

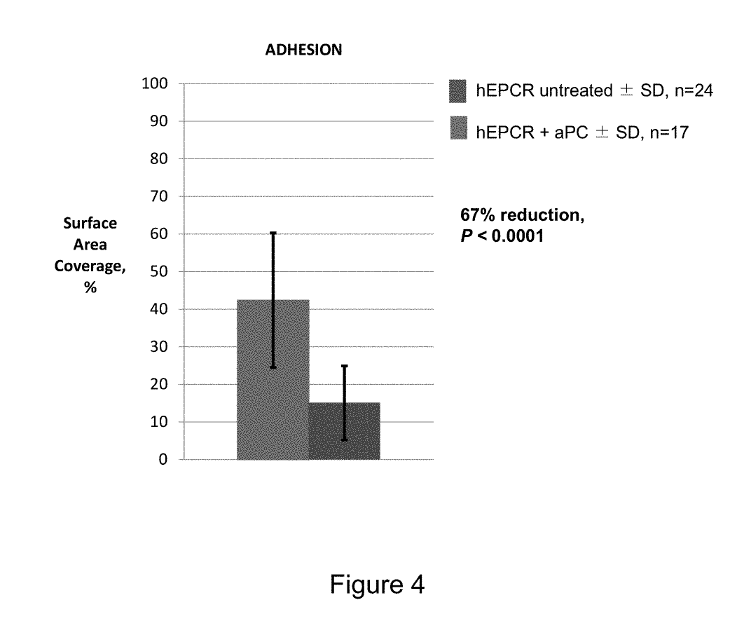

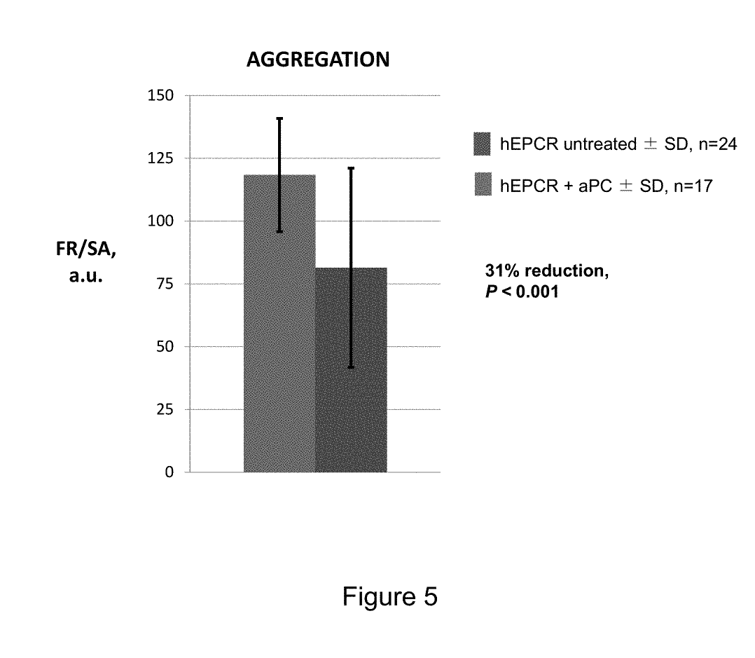

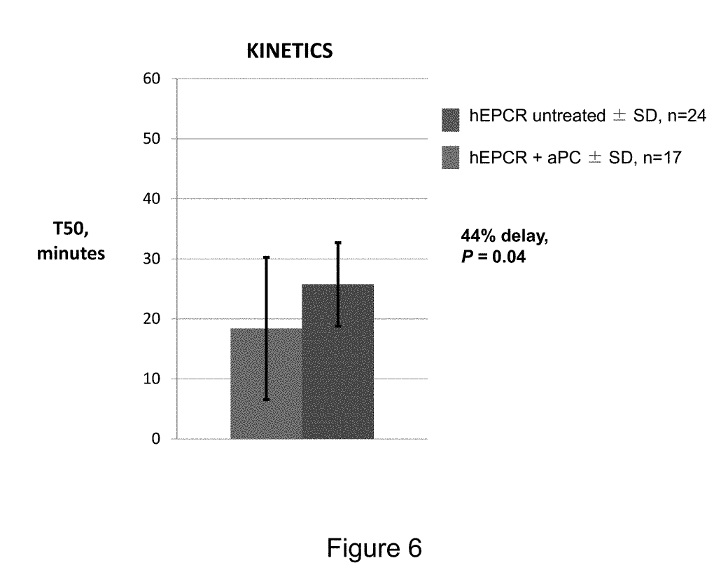

Adhesion and aggregation of untreated and aPC-treated hEPCR cells was examined. It was observed that treatment with aPC reduced significantly reduced adhesion (67% reduction; FIG. 4) and aggregation (31% reduction; FIG. 5). Further, it was observed that aPC treatment significantly delayed thrombosis (44% delay; FIG. 6).

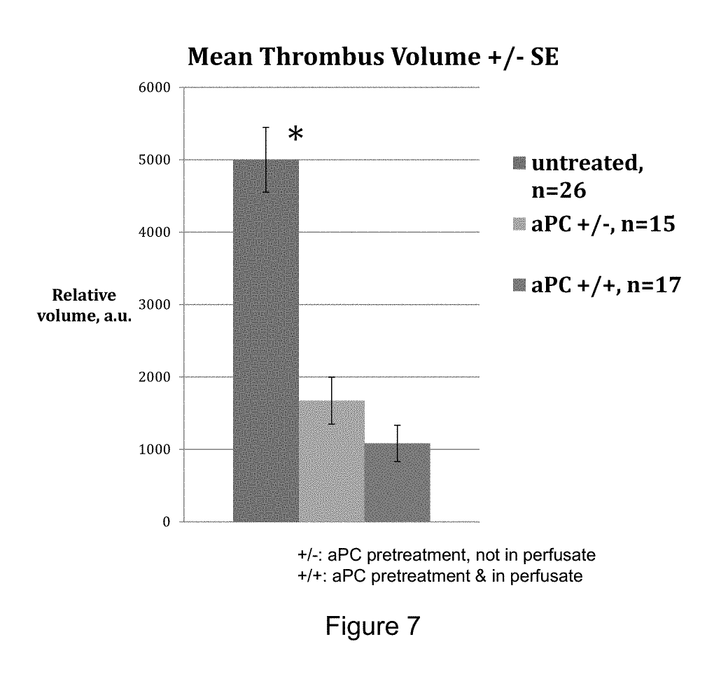

The volume of thrombus was evaluated in untreated cells, cells pretreated with aPC (but no aPC in perfusate), and cells pretreated with aPC (with aPC in perfusate). It was observed that the volume of the thrombus was significantly greater in untreated cells, and that most of the reduction in thrombosis seen with aPC treatment occurred from the pretreated cells, demonstrating that aPC pretreatment is cytoprotective (FIG. 7).

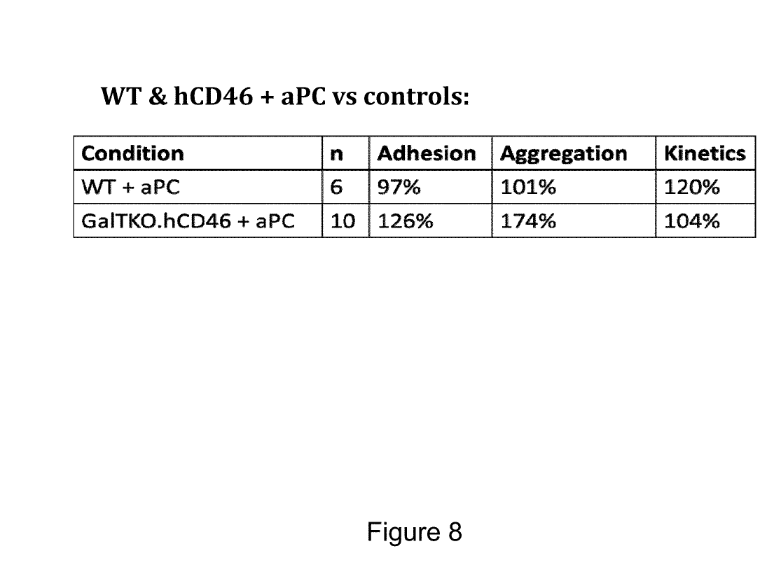

It was next examined whether the protective effects of aPC are specific to hEPCR+tissue. aPC treatment did not affect thrombosis on WT and hCD46 cells indicating that the aPC effects are specific to hEPCR+tissue (FIG. 8).

### Example 4

The experiments presented herein demonstrate that activated protein C modulates the thrombotic phenotype and vascular permeability of GalTKO.hCD46 porcine endothelium expressing human endothelial protein C receptor. Platelet activation, thrombin generation and increased vascular permeability are implicated in dysfunction of porcine lung xenografts, despite genetic modifications designed to control inflammation and thrombosis. It is demonstrated that that lungs from human endothelial protein C receptor (hEPCR) transgenic pigs have improved performance versus the GalKO.hCD46 background in pre-clinical models. Here, it is examined whether this protective phenotype may be mediated in part by anti-thrombotic or permeability blocking effects consequent to aPC binding to hEPCR.

Wild type (WT), GalKO.hCD46 and GalKO.hCD46.hEPCR porcine aortic endothelial cells (pAECs) were cultured in microfluidic channels. After confluent growth, control endothelia were incubated with media or aPC (0.02 μg/ml) for 6 hours and perfused with untreated blood (n=24). Perfusions were performed at 5 dynes/cm′ with heparinized human blood stained with calcein^{AM} to label platelets, and thrombus formation quantified over time. Vascular permeability was evaluated by impedance using xCELLigence system RTCA SP (Roche) using pAECs grown to confluence for 24 hours in presence of medium or rhaPC (1 mg/ml). Permeability was induced by thrombin (20 nM), and impedance (cell integrity index) measured continuously for another 24 hours.

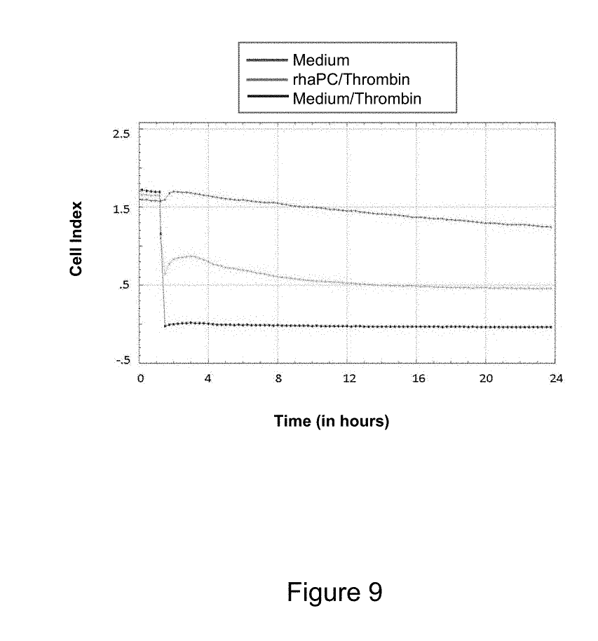

aPC treatment of GalKO.hCD46.hEPCR reduced (maximum percent of surface area [SA] covered with aPC: 15%±10, vs. no aPC: 42%±18; p<0.0001) and delayed adhesion (time to 50% maximal SA coverage [T₅₀]: 26±7 minutes, vs. 18±12; p=0.04), and attenuated platelet aggregation (fluorescence index: 81 units±40 vs. 118±23, p<0.001) relative to untreated GalKO.hCD46.hEPCR endothelia. aPC pre-treatment of GalTKO.hCD46 cells had no effect on either parameter of thrombus formation. The cell integrity index was stable overtime with medium only, but was dramatically decreased within 30 min by addition of thrombin. Pretreatment of EPCR expressing cells with aPC reduced the loss in cell barrier integrity by ˜50% (FIG. 9).

It is demonstrated herein that in physiologic assays, treatment with recombinant human aPC both reduced the pro-thrombotic GalKO.hCD46.hEPCR pAEC phenotype, and inhibited thrombin-induced increase in vascular permeability. These two independent mechanisms may explain the beneficial physiologic effects associated with hEPCR in lung models, an effect that appears to be specifically mediated by hEPCR expression, and suggest that pretreatment of organs with aPC may further enhance the performance of xenografts expressing hEPCR.

### Example 5

Experiments are conducted to examine the effect of pretreatment of allogenic and xenogeneic tissue with human activated protein C (aPC). Pretreatment of lungs with recombinant human activated protein C (aPC) is tested in both, allogeneic and xenogeneic perfusion settings.

Allogeneic Ex Vivo Perfusions:

In these perfusions, human lungs that were procured from deceased organ donors, but did not qualify for transplantation into a human recipient (e.g., due to infections or tumors) are cannulated and perfused (as a pair) on the Xvivo EVLP circuit for 4 hours. The applied perfusion methods are identical to clinical procedures previously described (Sanchez et al., 2012, J Heart Lung Transplant, 31(4): 339-48).

In two experiments, the lung pair is perfused with Steen solution to which aPC (0.02 μg/mL) is added as a single bolus at the beginning of the experiment. As a control, two more human lung perfusions are performed, in which no additional drug is added to the Steen solution.

In all four cases, lungs are administered Perfadex (plegia), cooled and taken off the Xvivo EVLP circuit after 4 hours. The lung pairs are surgically separated into left and right lungs and cannulated (pulmonary artery and main bronchus) individually. After a short ischemic time (<1 hour, for surgical separation and blood mixing), lungs (left and right) are then perfused separately with blood-type matching blood/plasma (˜1:1 ratio) perfusate for 8 hours or until lungs “fail” (e.g., no oxygenation, trachea edema, no blood flow through the lung).

During and at the end of the perfusion, blood samples and tissue samples are collected for later analyses. Functional lung parameters (pulmonary vascular resistance, airway pressure, blood oxygenation) are measured and recorded in real time.

Xenogeneic Ex Vivo Perfusions:

Porcine lungs, procured from transgeneic GalTKO.hCD46 pigs (provided by Revivicor), expressing human EPCR are cannulated and perfused (as a pair) on the Xvivo EVLP circuit for 4 hours. The applied perfusion methods are identical to clinical procedures previously described (Sanchez et al., 2012, J Heart Lung Transplant, 31(4): 339-48).

In two experiments, the lung pair is perfused with Steen solution to which aPC (0.02 μg/mL) is added as a single bolus at the beginning of the experiment. As a control, two more porcine lung perfusions are done, in which no additional drug is added to the Steen solution.

In all four cases, lungs are administered Perfadex (plegia), cooled and taken off the Xvivo EVLP circuit after 4 hours. The lung pairs are then surgically separated into left and right lungs and cannulated (Pulmonary artery and main bronchus) individually. After a short ischemic time (<1 hour, for surgical separation and blood mixing), lungs (left and right) are then perfused separately with blood-type matching blood/plasma (˜1:1 ratio) perfusate for 8 hours or until lungs “fail” (e.g. no oxygenation, trachea edema, no blood flow through the lung).

During and at the end of the perfusion, blood samples and tissue samples are collected for later analyses. Functional lung parameters (pulmonary vascular resistance, airway pressure, blood oxygenation) are measured and recorded in real time.

Xenogeneic In Vivo Lung Transplantation:

Multi-transgeneic porcine lungs (n=3), expressing human EPCR, are cannulated and perfused (as a pair) with Steen solution, containing aPC (as described above), on the Xvivo EVLP circuit for 4 hours. After the perfusion, the lungs are surgically separated. While the right lung is perfused according to the description provided in the xenogeneic ex vivo perfusion section, the left lung is transplanted into a left-sided pneumectomized recipient baboon. During and after the transplantation, lung biopsies and blood samples are collected for later analyses. As soon as the baboon is waking up from anesthesia and lung function is acceptable, the animal is extubated and monitored in its cage.

Analyses of Perfusions

The analyses of perfusion of allogeneic and xenogeneic tissue is similar. During the blood perfusion, blood and tissue samples are collected at pre-defined intervals for up to 8 hours or until the lungs fail. Biochemical analyses for blood cell counts (platelets, PMN), cell activation (CD62P, βTG, etc.), activation of the coagulation cascade (F1+2) and cytokine levels are compared between study and control groups.

Functional parameters, such as the pulmonary artery flow and pressure are recorded in real time and used to calculate the pulmonary vascular resistance (PVR). In addition, airway pressure and oxygenation (pO2) are measured. “Final” tissue is collected to measure the wet/dry weight ratio as an indicator for the tissue edema. Results, found in the both, allo- and xenogeneic aPC-pre-treatment perfusion experiments, are analyzed and compared to results obtained in experiments where no pre-treatment was performed.

Analyses of Xenogeneic Lung Transplantations

Blood, collected from the recipient during and after the transplantation, are used for biochemical analyses, as described above. In addition, the life-supportive function of the transplanted lung is assessed while the chest is still open by obstructing the right pulmonary artery, only allowing blood flow through the transplanted lung. Imaging techniques (X-ray, CT) are used to investigate lung function and condition in the postoperative time. Experiments, in which the lung was pre-treated with aPC, are compared to experiments, in which lungs did not receive the aPC pre-treatment but otherwise similar drug regimen (n>40 already performed).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method for improving the viability of transplant graft comprising contacting the transplant graft with a composition comprising at least one selected from the group consisting of activated Protein C (aPC), an isolated nucleic acid molecule encoding aPC, Protein C (PC), and an isolated nucleic acid molecule encoding PC.

2. The method of claim 1, wherein the transplant graft is contacted with the composition ex vivo.

3. The method of claim 1, wherein the transplant graft is allogeneic.

4. The method of claim 1, wherein the transplant graft is xenogeneic.

5. The method of claim 1, wherein the transplant graft expresses Endothelial Protein C Receptor (EPCR).

6. The method of claim 5, wherein the transplant graft is from an organism modified to express EPCR.

7. The method of claim 1, wherein the transplant graft is selected from the group consisting of lung, heart, kidney, liver, pancreas, intestine, and multivisceral transplant.

8. The method of claim 1, wherein the method comprises perfusing the transplant graft with the composition.

9. The method of claim 1, wherein the composition comprises a crystalloid perfusate comprising aPC.

10. The method of claim 1, wherein the composition comprises a whole-blood perfusate comprising aPC.

11. The method of claim 1, wherein the method reduces or prevents thrombosis in the transplant graft.

12. A method for improving the viability of transplant graft comprising:
a.) obtaining transplant graft;
b.) contacting the transplant graft with a composition comprising at least one selected from the group consisting of activated Protein C (aPC), an isolated nucleic acid molecule encoding aPC, Protein C (PC), and an isolated nucleic acid molecule encoding PC; and
c.) transplanting the transplant graft to a recipient.

13. A composition for improving the viability of transplant graft comprising at least one of the group consisting of activated Protein C (aPC), an isolated nucleic acid molecule encoding aPC, Protein C (PC), and an isolated nucleic acid molecule encoding PC.

14. The composition of claim 12, wherein the composition comprises a crystalloid perfusate.

15. The composition of claim 12, wherein the composition comprises a whole-blood perfusate.
